(19) United States
(12) Patent Application Publication
(10) Pub. No.: US 2025/0250495 A1
(43) Pub. Date: Aug. 7, 2025

(54) PROCESS FOR PRODUCTION OF A LIQUID FUEL FROM OXYGENATES

(71) Applicant: UOP LLC, Des Plaines, IL (US)

(72) Inventors: Joel S. Paustian, Vernon Hills, IL (US); Manuela Serban, Northbrook, IL (US); Eseoghene Jeroro, Chicago, IL (US); Andrew Martin Meloan, Chicago, IL (US); Martha Leigh Abrams, Chicago, IL (US)

(21) Appl. No.: 19/047,600

(22) Filed: Feb. 6, 2025

Related U.S. Application Data
(60) Provisional application No. 63/550,585, filed on Feb. 6, 2024.

(51) Int. Cl. C10G 69/12

(52) U.S. Cl. CPC C10G 69/126; C10G 2300/1011; C10G 2400/20

(57) ABSTRACT

A process for production of a liquid fuel is disclosed. The process comprises producing a ethylene stream in a dehydration unit. A C3 olefin stream is produced in a MTO unit. The C3 olefin stream and the ethylene stream are oligomerized with an oligomerization catalyst to produce an oligomerized olefin stream comprising a fuel stream.

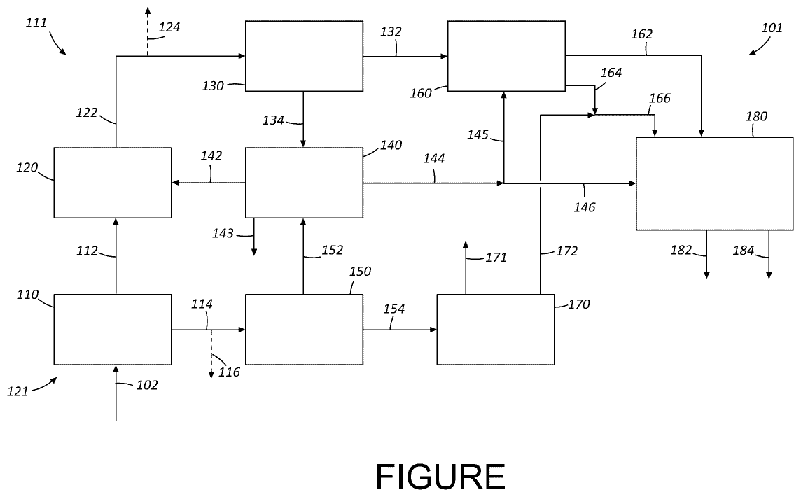

### FIELD

The field is the production of fuel from biorenewable feedstock. The field may particularly relate to an integrated production of fuel from alcohols.

### BACKGROUND

As the demand for fuel increases worldwide, there is increasing interest in producing fuels and blending components from sources other than crude oil. Often referred to as biorenewable sources, these sources include, but are not limited to, plant oils such as corn, rapeseed, canola, soybean, microbial oils such as algal oils, animal fats such as inedible tallow, fish oils and various waste streams such as yellow and brown greases and sewage sludge. A common feature of these sources is that they are composed of glycerides and free fatty acids (FFA). Both triglycerides and the FFAs contain aliphatic carbon chains having from about 8 to about 24 carbon atoms. The aliphatic carbon chains in triglycerides or FFAs can be fully saturated, mono, di or poly-unsaturated.

In a bio-refinery, a large amount of renewable-based carbon monoxide and carbon dioxide are produced in the cracking and hydroprocessing reactors from thermal or catalytic deoxygenation of biomass to produce oxygen-free biofuels, including sustainable aviation fuel, bio-gasoline, bio-diesel and bio-marine fuel. In addition, renewable-based carbon monoxide and carbon dioxide are also produced in the regenerator from the burning of coke on spent catalysts and char produced in the reactor due to limited mass and heat transfer, while the energy generated supplies the heat balance in the process. The total amount of carbon in the biogenic carbon monoxide and carbon dioxide generated in the process could account for up to 50% of the carbon from renewable biomass feed. It remains a challenge to cost-effectively capture the renewable-based carbon monoxide and carbon dioxide and convert them to more valuable products.

Great importance has been attached to renewable energy resources all over the world. Biomass derived ethanol fuel is becoming more common, and the processes of producing ethanol fuel from starch and cellulose are undergoing continuous improvement. Fuels originating from biological sources are known as biogenic fuels.

Bioethanol can be produced by fermentation of biological feedstock. Fermentation produces substantial carbon dioxide which must be managed. The bioethanol is then dehydrated to produce ethylene.

An ethanol to jet fuel process is one of the routes that holds promise to minimize or eliminate net carbon combustion. The end product of this process is jet and diesel fuel produced from bioethanol. The resulting jet fuel is a sustainable aviation fuel (SAF) intended to replace jet fuel produced from conventional sources such as crude oil.

Jet fuel is one of the few petroleum fuels that cannot be replaced easily by electrical motor systems because a high energy density is required to fuel planes which cannot be supplied with electric batteries. Large incentives are currently available for green jet fuel in certain regions to reduce the environmental impact of fossil-derived jet fuels.

Low carbon intensive SAF is needed to reduce aviation greenhouse gas emissions. SAF also diminish dependence on fossil fuels. Furthermore, existing routes to produce SAF are cost intensive and feedstock limited.

As refiners seek to add capability for processing biorenewable feedstocks, processes are sought to produce greater volumes of jet fuel due to its high value and demand. Processes for producing fuel from biorenewable feedstocks are desired.

### SUMMARY

A process for production of a liquid fuel is disclosed. The process comprises producing a C2 olefin stream in a dehydration unit. A C3 olefin stream is produced in a MTO unit. The C3 olefin stream and the C2 olefin stream are oligomerized with an oligomerization catalyst to produce an oligomerized olefin stream boiling in the fuel range. The process integrates an MTO unit and an ethanol dehydration unit to convert the biogenic carbon oxides into liquid fuel.

### BRIEF DESCRIPTION OF THE DRAWINGS

The FIGURE is a simplified process flow diagram of the present disclosure.

### DEFINITIONS

The term “communication” means that material flow is operatively permitted between enumerated components.

The term “downstream communication” means that at least a portion of material flowing to the subject in downstream communication may operatively flow from the object with which it communicates.

The term “upstream communication” means that at least a portion of the material flowing from the subject in upstream communication may operatively flow to the object with which it communicates.

The term “direct communication” means that flow from the upstream component enters the downstream component without passing through a fractionation or conversion unit to undergo a compositional change due to physical fractionation or chemical conversion.

The term “indirect communication” means that flow from the upstream component enters the downstream component after passing through a fractionation or conversion unit to undergo a compositional change due to physical fractionation or chemical conversion.

The term “bypass” means that the object is out of downstream communication with a bypassing subject at least to the extent of bypassing.

The term “column” means a distillation column or columns for separating one or more components of different volatilities. Unless otherwise indicated, each column includes a condenser on an overhead of the column to condense and reflux a portion of an overhead stream back to the top of the column and a reboiler at a bottom of the column to vaporize and send a portion of a bottoms stream back to the bottom of the column. Feeds to the columns may be preheated. The top pressure is the pressure of the overhead vapor at the vapor outlet of the column. The bottom temperature is the liquid bottom outlet temperature. Overhead lines and bottoms lines refer to the net lines from the column downstream of any reflux or reboil to the column. Stripper columns may omit a reboiler at a bottom of the column and instead provide heating requirements and separation impetus from a fluidized inert media such as steam. Stripping columns typically feed a top tray and take main product from the bottom.

As used herein, the term “a component-rich stream” means that the rich stream coming out of a vessel has a greater concentration of the component than the feed to the vessel.

As used herein, the term “a component-lean stream” means that the lean stream coming out of a vessel has a smaller concentration of the component than the feed to the vessel.

As used herein, the term “boiling point temperature” means atmospheric equivalent boiling point (AEBP) as calculated from the observed boiling temperature and the distillation pressure, as calculated using the equations furnished in ASTM D86 or ASTM D2887.

As used herein, the term “True Boiling Point” (TBP) means a test method for determining the boiling point of a material which corresponds to ASTM D-2892 for the production of a liquefied gas, distillate fractions, and residuum of standardized quality on which analytical data can be obtained, and the determination of yields of the above fractions by both mass and volume from which a graph of temperature versus mass % distilled is produced using fifteen theoretical plates in a column with a 5:1 reflux ratio.

As used herein, the term “T5” or “T95” means the temperature at which 5 mass percent or 95 mass percent, as the case may be, respectively, of the sample boils using ASTM D-86 or TBP.

As used herein, the term “initial boiling point” (IBP) means the temperature at which the sample begins to boil using ASTM D2887, ASTM D-86 or TBP, as the case may be.

As used herein, the term “end point” (EP) means the temperature at which the sample has all boiled off using ASTM D2887, ASTM D-86 or TBP, as the case may be.

As used herein, the term “diesel boiling range” means hydrocarbons boiling in the range of an IBP between about 125° C. (257° F.) and about 175° C. (347° F.) or a T5 between about 150° C. (302° F.) and about 200° C. (392° F.) and the “diesel cut point” comprising a T95 between about 343° C. (650° F.) and about 399° C. (750° F.) using the TBP distillation method.

As used herein, the term “diesel conversion” means conversion of feed that boils above the diesel cut point to material that boils at or below the diesel cut point in the diesel boiling range.

As used herein, the term “separator” means a vessel which has an inlet and at least an overhead vapor outlet and a bottoms liquid outlet and may also have an aqueous stream outlet from a boot. A flash drum is a type of separator which may be in downstream communication with a separator that may be operated at higher pressure.

As used herein, the term “predominant” or “predominate” means greater than 50%, suitably greater than 75% and preferably greater than 90%.

As used herein, the term “Cₓ” are to be understood to refer to molecules having the number of carbon atoms represented by the subscript “x”. Similarly, the term “Cₓ−” refers to molecules that contain less than or equal to x and preferably x and less carbon atoms. The term “Cₓ+” refers to molecules with more than or equal to x and preferably x and more carbon atoms.

As used herein, the term “carbon number” refers to the number of carbon atoms per hydrocarbon molecule and typically a paraffin molecule.

As used herein, the term “passing” includes “feeding” and means that the material passes from a conduit or vessel to an object.

As used herein, the term “rich” can mean an amount of at least generally 50%, and preferably 70%, more preferably 90% or above by mass of a compound or class of compounds in a stream.

As used herein, the term “biofuel,” as defined herein, is a fuel product at least partly derived from “biomass,” the latter being a renewable resource of biological origin.

### DETAILED DESCRIPTION

Ethanol producers are striving to meet the future market demands for sustainable aviation fuel (SAF) production with minimum carbon intensity and capital expenditure. A 100% biogenic carbon dioxide stream offers an opportunity for reduction in carbon intensity. Stakeholders may not have access to carbon dioxide sequestration infrastructure and cannot expect timely pipeline access. The present disclosure provides an integrated solution to convert carbon dioxide onsite to liquid fuel products, which can be easily transported with pre-existing infrastructure. The present disclosure provides an integrated process for an MTO unit and an ethanol dehydration unit to reduce capital expenditure and complexity compared to the standalone technologies while valorizing the carbon dioxide into an attractive and actionable product package.

In the FIGURE, in accordance with an exemplary embodiment, a process 101 is shown for production of a liquid fuel. The process 101 comprises an ethanol production unit 121, a methanol to olefins (MTO) unit 111, an electrolyzer 140, and an oligomerization unit 180. In an exemplary embodiment, the MTO unit 111 comprises a methanol synthesis section 120, a MTO reaction section 130, and an olefins recovery section 160. In another exemplary embodiment, the ethanol production unit 121 comprises an ethanol plant 110, an ethanol dehydration unit 150, and an ethylene purification section 170.

In the ethanol plant 110, a feed of biomass is fermented into alcohol. In an exemplary embodiment, the biomass feedstock may be corn. Other biomass feed stocks are envisioned. In the embodiment, corn is delivered in line 102 to a storage bin perhaps a silo. Grit and stones are removed and the biomass is milled into a flour and slurried with an enzyme such as alpha amylase in aqueous lime to hydrolyze α-bonds of large, a-linked polysaccharides, such as starch and glycogen, yielding shorter chains thereof, dextrins, and maltose. The slurry is then heated and stirred in a liquefaction tank to provide a mash which is then cooked in the presence of sulfuric acid to break up polymeric lignin and cellulose. Glucoamylase is added to the cooked mash to saccharify the mash under heating and stirring to produce dextrin. Nutrients and antifoaming agents are added to the saccharified mash to ferment the saccharides to alcohol including ethanol. Carbon dioxide is produced during fermentation, some of which can be recycled back to the fermentation process to assist in agitation, but a net carbon dioxide stream is produced in line 112.

Dilute ethanol is concentrated first in a beer still and then a condensed overhead stream is taken to a rectifier column to concentrate the ethanol stream to about 90%. The beer still is operated at around atmospheric pressure with a bottoms temperature of about 90° C. (194° F.) to about 110° C. (230° F.). The rectifier column is operated at just above atmospheric pressure with a bottoms temperature of about 90° C. (194° F.) to about 110° C. (230° F.). The rectifier column is operated with an overhead pressure of about 350 to about 450 kPa (gauge).

The concentrated ethanol stream from the rectifier overhead may be transported in line 114 to the ethanol dehydration unit 150. Alternatively, ethanol can be imported into the process instead of being provided from the ethanol production unit 121. Moreover, some of the ethanol in line can be exported from the process in line 116 to be utilized elsewhere. The ethanol stream in line 114 may be heated and charged to an ethanol dehydration reactor in the vapor phase at a temperature of about 400° C. to about 550° C. and a pressure of about 455 kPa (gauge) 65 psig to about 630 kPa (gauge) (90 psig) to produce ethylene and water. The ethylene product stream may be quenched with water and compressed to about 455 kPa (gauge) (165 psig) to about 3220 kPa (gauge) (460 psig) in two stages and water washed to absorb oxygenates from the ethylene stream and dried to remove water. Water may be recycled to the quench and the water wash vessels, while net water is removed from the ethanol dehydration unit 150 in line 152 which may be transported to the electrolyzer 140. The dried ethylene stream may then be fed to a heavy oxygenates removal column to obtain a deoxygenated ethylene stream in the overhead while heavy oxygenates are removed in a bottom stream. The heavy oxygenate removal column may be operated with a bottom temperature of about −29° C. (−20° F.) to about 121° C. (250° F.) and a pressure of about 2.4 MPa (gauge) (350 psig) to about 3.1 MPa (gauge) (450 psig) in the overhead. The ethylene stream may then be further compressed and transported in line 154 to the ethylene purification section 170.

Ethylene in line 154 contains carbon monoxide and water which can be odious to the oligomerization catalyst. Hence, the ethylene stream in line 154 may be fractionated in an ethylene column to separate carbon monoxide in an overhead stream from ethylene in a bottom stream. The ethylene column may be operated with a bottom temperature of about −45° C. (−50° F.) to about −29° C. (−20° F.) and a pressure of about 2.76 MPa (gauge) (400 psig) to about 3.45 MPa (gauge) (500 psig) in the overhead. Alternatively, carbon monoxide may be adsorbed from the ethylene stream in a regenerable adsorbent bed comprising a copper-based adsorbent.

The ethylene in the bottoms may contain moisture which can be removed in an adsorbent bed comprising a synthetic crystalline aluminosilicate or another hydrophilic adsorbent. A purified ethylene stream may be recovered in line 172 and combined with a concentrated vaporous C2 olefin stream in line 164 to provide a combined C2 olefin stream in line 166. The purified ethylene stream in line 172 is large and may be combined with the concentrated vaporous C2 olefin stream in line 164 downstream of the light olefins recovery section 160 to reduce the capacity required in the light olefins recovery section.

It is contemplated that other alcohols can be produced in the ethanol plant 110 in which case other olefins would be produced in the dehydration unit 150 and provided in line 172.

Carbon dioxide is a so-called greenhouse gas which concentration many desire to suppress in the atmosphere. The carbon dioxide stream in line 112 is large and continuous. Many would resort to suppressing it and storing it. However, carbon dioxide may be converted to oxygenates such as methanol or dimethyl ether. Molecular sieves such as microporous crystalline zeolite and non-zeolitic catalysts, particularly silicoaluminophosphates (SAPO), are known to promote the conversion of oxygenates to hydrocarbon mixtures, particularly hydrocarbon mixtures composed largely of light olefins. So, we propose to utilize the abundant carbon dioxide to produce methanol that can be employed to produce olefins. Accordingly, the carbon dioxide stream in line 112 is transported to the methanol synthesis section 120.

The net carbon dioxide stream in line 112 from the fermentation section in the ethanol plant 110 and a hydrogen gas stream in line 142 from the electrolyzer 140 are passed to the methanol synthesis section 120. In an aspect, the carbon dioxide stream in line 112 may be taken from any suitable source. In accordance with the present disclosure, the carbon dioxide stream in line 112 is taken from the ethanol plant 110.

In another aspect, a supplemental syngas stream may be passed to the methanol synthesis section 120. In accordance with an exemplary embodiment, of the present disclosure, the methanol synthesis section 120 may comprise one or more methanol converter(s). The carbon dioxide stream in line 112 and the hydrogen gas stream in line 142 are passed to the methanol converter of the methanol synthesis section 120. In an embodiment, the carbon dioxide stream in line 112 and the hydrogen gas stream in line 142 may be combined to provide a combined feed stream which is passed to the methanol converter. In an aspect, the carbon dioxide stream in line 112 may be compressed before passing it to the methanol converter in the methanol synthesis section 120.

In the methanol converter of the methanol synthesis section 120, the carbon dioxide and hydrogen react and are converted to methanol and water. The methanol synthesis process is accomplished in the presence of a methanol synthesis catalyst. A suitable methanol synthesis catalyst may be a copper on a zinc oxide and alumina support. Synthesis conditions in the methanol converter of the methanol synthesis section 120 may include a temperature of about 200° C. to about 300° C. and a pressure of about 3.5 MPa to about 10 MPa. Reaction equilibrium typically requires methanol separation and recycle of unreacted reagents to the synthesis reaction.

The methanol synthesis reaction in the methanol synthesis section 120 is highly exothermic. A boiler feed water stream may be passed to the methanol converter to absorb exothermic heat by indirect heat exchange and generate a steam stream. The steam stream may be used to provide heat elsewhere in the process 101.

The methanol stream from the methanol converter may also comprise methanol, dimethyl ether, ethanol or combinations thereof. The methanol stream from the methanol converter may be cooled by heat exchange and separated into a vapor stream and a liquid stream. The vapor stream may comprise unconverted carbon dioxide. The vapor stream may be recycled back to the methanol converter and converted to methanol and other oxygenates. The liquid stream may be a crude methanol stream. In an aspect, the crude methanol stream may comprise at least 100 ppmw of carbon oxide and/or at least 100 ppmw C2+ oxygenates.

The crude methanol stream may be passed to a crude methanol hold-up tank in the methanol synthesis unit 120. The crude methanol stream may be taken from the crude methanol hold-up tank and passed to a methanol purification column to remove by-products and provide a purified methanol stream. The methanol purification column may comprise one or more distillation columns. In the methanol purification column, light gas(es) is separated from the crude methanol stream. The light gases separated from the crude methanol stream may include carbon monoxide, carbon dioxide, methane, and hydrogen. The crude methanol stream may comprise heavy oxygenates such as C2+ alcohols, ketones, and aldehydes which should be removed from the purified methanol stream in a methanol rectifier column. The heavy oxygenates are separated from the purified methanol stream in a methanol rectifier column to produce a methanol product stream in a rectifier overhead line of the methanol rectifier column. The methanol product stream may be heat exchanged with the purified methanol stream and passed to a methanol product hold-up tank. The methanol product stream of the methanol synthesis unit may be taken in line 122 for further processing.

In accordance with an exemplary embodiment, the methanol purification column and the methanol rectification column may be operated at a pressure from about 689 kPa (100 psia) to about 1379 kPa (200 psia). In accordance with another exemplary embodiment, the distillation column may be operated at a temperature of about 27° C. (80° F.) to about 177° C. (350° F.).

The highly efficient methanol to olefin (MTO) process may convert oxygenates such as methanol to light olefins which has been typically employed for plastics production. Methanol and dimethyl ether are converted into light olefin products in the MTO process. Molecular sieves such as microporous crystalline zeolite and non-zeolitic catalysts, particularly silicoaluminophosphates (SAPO), promote the conversion of oxygenates such as methanol to hydrocarbon mixtures composed largely of light olefins. SAPO catalysts and their formulation are generally taught in U.S. Pat. Nos. 4,499,327A, 10,358,394 and 10,384,986. Light olefins produced from the MTO process are concentrated in ethylene and propylene but include C4-C6 olefins.

Turning to the FIGURE, the methanol product stream may be taken in line 122 from the methanol synthesis section 120 and passed to a reactor of the MTO reaction section 130. Optionally, a portion of the methanol product stream may be taken in line 124 and exported. Alternatively, the methanol product stream in line 122 may be imported into the process from an external source to supplement or provide all of the product methanol stream in line 122.

The methanol product stream of oxygenates may be superheated and charged to an oxygenate conversion reactor of the MTO reaction section 130 that reacts the stream of oxygenates comprising the methanol stream in line 122 and/or dimethyl ether (DME) with a fluidized catalyst. The MTO reaction conditions include contact with a SAPO catalyst at a pressure between about 2 MPa and about 3.8 MPa. The MTO reaction temperature should be between about 325° C. to about 450° C. A weight hourly space velocity (WHSV) in the oxygenate conversion reactor is in the range of about 2 to about 15 hr−1. The MTO catalyst is separated from the product olefin stream downstream the MTO reaction.

The hot vaporous MTO reactor effluent stream may be preliminarily cooled in a reactor effluent heat exchanger to recover heat before it is passed to a quench tower. In the quench tower, the vaporous reactor effluent is desuperheated, neutralized of organic acids and clarified of catalyst fines by direct contact with a water stream which may be taken from a stripped water stream. A quenched reactor effluent stream is separated in one or more stripper sections to generate a product olefin stream and a product water stream comprising oxygenate byproducts from the quenched reactor effluent stream. An overhead product olefin stream comprising olefins from a product separator column in line 132 is taken from the MTO reactor section 130 and delivered to a compression train of the light olefins recovery section 160. The product water stream includes dilute hydrocarbon oxygenates such as DME, methanol, acetaldehyde, acetone and methylethylketone (MEK). A water stripper column separates or strips the oxygenates into an oxygenate rich stream rich in both methanol and oxygenates and a water rich stream which can be recirculated for water needs in the MTO reactor section 130.

The product olefin stream carries valuable olefinic products which must be recovered. The compression section in the light olefins recovery section 160 increases the pressure of the product olefin stream necessary for olefin recovery. The compression section may comprise one or more knock out drums which separates the product olefin stream into a pressurized olefin rich stream at a temperature of about 40° C. (104° F.) to about 60° C. (140° F.) and a pressure of about 193 kPa (g) (28 psig) to about 262 kPa (g) (38 psig) and an aqueous stream rich in oxygenates. The aqueous stream is returned with the product water stream to the water stripper column.

At least a portion of the compressed product stream is contacted in an oxygenate absorber column at effective conditions to absorb at least a quantity of effluent oxygenates with a cooled lean water stream. Contacting in the oxygenate absorber column produces an absorption olefin rich stream and an absorption water rich stream comprising a quantity of effluent oxygenates. The oxygenate absorber may have operating conditions including a bottoms temperature range of about 30° C. (86° F.) to about 60° C. (140° F.) and an overhead pressure range of about 700 kPa gauge (101 psig) to about 1 MPa gauge (145 psig).

The absorption olefin rich stream may be fed to a stripper separator which separates an aqueous stream including oxygenates in the boot, a light olefinic vapor stream comprising C3− olefins and a heavy olefinic liquid stream comprising C4+ olefins. The heavy olefinic liquid stream is stripped in a DME stripper column to remove C3− vapors from the heavy olefinic liquid stream in the stripper bottoms stream. The bottom stream comprises mostly C4+ olefins but comprises diolefins that will deter the oligomerization catalyst requiring selective hydrogenation. The stripper separator may operate at a temperature of about 30° C. (86° F.) to about 60° C. (140° F.) and a pressure of about 1.7 MPa(g) (250 psig) to about 2.1 MPa(g) (300 psig). The light olefinic vapor stream in the overhead of the DME stripper column is scrubbed in a caustic scrubber column by contact with a caustic solution to absorb acid gases such as carbon dioxide from the light olefinic vapor which exits the caustic scrubber.

The scrubbed light olefinic vapor in the overhead may be refrigerated, for example in a drier feed chiller to liquefy part of the light olefinic stream and separated in a drier separator to provide an aqueous stream from a boot and a vaporous light olefin stream comprising C2− hydrocarbons and gases in the overhead and a liquid light olefin stream comprising C3+ hydrocarbons in the bottoms. The vaporous light olefin stream is dried in an adsorbent drier to remove moisture and provide a dried vaporous olefin stream. The dried vaporous olefin stream may comprise C2 olefins. The liquid light olefin stream is also dried in a separate adsorbent drier to remove moisture and provide a dried liquid olefin stream which is a C3+ olefin stream. The dried liquid olefin stream may comprise C3 to C6 olefins.

The light olefins recovery section 160 comprises an oligomerization feed preparation section. The oligomerization feed preparation section comprises a fractionation section, a selective hydrogenation reactor, and an oxygenate removal unit. The fractionation section may comprise a demethanizer fractionation column, a deethanizer fractionation column, a water wash column, and a DME wash water stripper column. The dried vaporous olefin stream and the dried liquid olefin stream are passed to the demethanizer fractionation column. The vaporous C2 olefin stream and the C3+ olefin stream are fractionated in the demethanizer fractionation column to provide an overhead light gas stream and a bottom demethanized C2+ stream. The demethanizer bottom temperature may be about 0° C. (32° F.) to about 45° C. (113° F.) and the demethanizer overhead pressure of about 2.4 MPa(g) (350 psig) to about 3.5 MPa(g) (500 psig).

The demethanized C2+ stream is deethanized by fractionation in the deethanizer column to provide an ethylene stream in the overhead and a bottom deethanized C3+ stream. The deethanizer column may be operated at a bottom temperature of about 43° C. (110° F.) to about 104° C. (220° F.) and an overhead pressure of about 1.8 MPa(g) (260 psig) to about 3.2 MPa(g) (460 psig). The ethylene overhead stream is condensed to provide a net overhead liquid stream which is combined with a hydrogen stream and passed to an acetylene conversion reactor to convert acetylenes to ethylene and produce a concentrated ethylene stream. The acetylene conversion catalyst may be a palladium and silver on aluminum oxide catalyst. The acetylene conversion conditions may include a pressure of about 1.4 MPa(g) (200 psig) to about 2.8 MPa(g) (400 psig) and a temperature of about 38° C. (100° F.) to about 93° C. (200° F.). The concentrated vaporous C2 olefin stream may be dried and produced in line 164.

The deethanized C3+ stream is routed to the water wash column to absorb oxygenates to provide an oxygenate rich water wash stream and a washed deethanized olefin rich stream. A water wash stream from a DME wash water stripper column is routed to the water wash column and contacted with the deethanized C3+ stream to absorb the oxygenates. Suitably, the washed deethanized C3+ stream in the overhead has a total oxygenate concentration of no more than 500 wppm. The water wash column may be operated at a bottom temperature of about 10° C. (50° F.) to about 66° C. (150° F.) and an overhead pressure of about 2.4 MPa (gauge) (350 psig) to about 3.2 MPa (gauge) (450 psig).

The oxygenate rich water wash stream from the bottoms of the water wash column is passed to the DME wash water stripper column to be stripped of DME and other oxygenates to produce an overhead DME stream which may be recycled to the MTO reactor in the MTO reactor section 130. A stripped water wash stream from the bottom of the DME wash water stripper column is recycled to the water wash column perhaps after supplementation with a make-up water stream. The overhead washed deethanized olefin rich stream from the water wash column comprising C3 to C8 olefins also contains diolefins that could cause cross-link polymerization in the oligomerization reactor, so it is combined with a hydrogen stream and charged to the selective hydrogenation reactor. In the selective hydrogenation reactor, diolefins and residual acetylenes are converted to mono-olefins to provide a mono-olefin stream. The selective hydrogenation reactor is operated in liquid phase at pressures include about 2.3 MPa(g) (330 psig) to about 3.1 MPa(g) (450 psig) and a temperature between about 20° C. (68° F.) and about 100° C. (212° F.). The liquid hourly space velocity of the reactants through the selective hydrogenation catalyst should be above about 1.0 hr⁻¹ and below about 35.0 hr⁻¹, and the mole ratio of hydrogen to diolefinic hydrocarbons in the selective hydrogenation reactor charge is maintained between 1:1 and 4.5:1. Suitable catalysts include, but are not limited to, a catalyst comprising copper and at least one other metal such as titanium. The metals are preferably supported on an inorganic oxide support such as silica and alumina, for example. The mono-olefin stream may comprise an acetylene and diolefin concentration of no more than about 50 to about 80 wppm.

The mono-olefin stream may be transported to the oxygenate removal unit to adsorb residual oxygenates including DME, water, and other trace oxygenates. The deoxygenated olefin stream comprises C3 to C8 olefins and not more than 1 wppm oxygenate including DME and water. The oxygenate removal unit may be operated at an inlet temperature of about 10° C. (50° F.) to about 66° C. (150° F.) and an inlet pressure of about 2.3 MPa (gauge) (330 psig) to about 3 MPa (gauge) (430 psig). The adsorbent in the oxygenate removal unit may be a large pore molecular sieve. A deoxygenated liquid C3 olefin stream in line 162 may be charged to an oligomerization reactor in the oligomerization unit 180. In an exemplary embodiment, the dried concentrated ethylene stream may be combined with a dehydrated C2 olefin stream in line 172 from the ethanol dehydration unit 150 to provide a combined C2 olefin stream in line 166 which is charged to the oligomerization reactor in the oligomerization unit 180.

A deoxygenated liquid C3 olefin stream is taken in line 162 and passed to the oligomerization unit 180. In accordance with the present disclosure, the concentrated vaporous C2 olefin stream in line 164 is also passed to the oligomerization unit 180 perhaps after combination with the purified ethylene stream in line 172 to provide the combined C2 olefin stream in line 166.

The electrolyzer 140 can take the various water streams that are generated during the separation steps and produce hydrogen streams which can be fed to the MTO unit 111, the ethanol production unit 121, and the oligomerization unit 180. Referring to the electrolyzer 140, a water stream in line 134 from the water stripper of the MTO reaction section 130 may be passed to the electrolyzer 140. Another water stream from the light olefins recovery section 160 may also be passed to the electrolyzer 140. Heat is also provided to the electrolyzer 140 from any suitable heat source. In an aspect, the electrolyzer 140 is powered by green energy. In accordance with the present disclosure, the water stream in line 152 produced in the dehydration unit 150 is also passed to the electrolyzer 140. Various types of electrolyzers may be used as the electrolyzer 140 including but not limited to a polymer electrolyte membrane/proton exchange membrane (PEM/PEMEC), an alkaline electrolysis cell (AEC), an anion exchange membrane (AEM), and a solid oxide electrolysis cell (SOE/SOEC).

An oxygen stream is withdrawn in line 143 from the electrolyzer 140. The oxygen stream in line 143 may be vented, recovered as byproduct, or used for synthetic air generation for MTO regenerator and/or fired heaters. Hydrogen produced in the electrolyzer 140 can be separated to provide the first hydrogen gas stream in line 142 that is employed in the methanol synthesis section 120. A second hydrogen stream is provided in line 144 from the electrolyzer 140. In an aspect, the second hydrogen stream in line 144 may be separated into a third hydrogen stream in line 145 and a fourth hydrogen stream in line 146. The third hydrogen stream in line 145 may be passed to the light olefin recovery section 160 to provide hydrogen to the acetylene conversion reactor and the selective hydrogenation reactor of the oligomerization feed preparation section. The fourth hydrogen stream in line 146 may be passed to the oligomerization unit 180 for the hydrogenation of olefins.

In the oligomerization unit 180 the combined C2 olefin stream in line 166 and the deoxygenated liquid C3 olefin stream in 162 are oligomerized together or perhaps separately into oligomers. The combined C2 olefin stream in line 166 and the deoxygenated liquid C3 olefin stream in line 162 may be mixed with a hydrocarbon diluent which may be a recycled diesel range diluent produced from the oligomerization process and charged to the oligomerization reactors. The combined ethylene stream and the deoxygenated liquid olefin stream may be split and charged to a series of oligomerization reactors. Suitably, the charge olefin stream may be first charged to a series of first stage oligomerization reactors. The combined ethylene stream and the deoxygenated liquid olefin stream may be charged to the first stage oligomerization reactors at a temperature of about 180° C. (356° F.) to about 230° C. (446° F.) and a pressure of about 3.5 MPag (500 psig) to about 8.4 MPag (1200 psig). Effluent from one first stage oligomerization reactor may be cooled, mixed with another portion of the charge olefin stream and/or a recycle olefin stream and/or diluent stream and charged to a subsequent first stage oligomerization reactor. The first stage oligomerization reactors may operate at a temperature of about 180° C. (356° F.) to about 230° C. (446° F.) and a pressure of about 3.5 MPag (500 psig) to about 8.4 MPag (1200 psig). Oligomerate from the first stage oligomerization reactors may be charged to one or a series of second stage oligomerization reactors to further oligomerize the first stage oligomerate or to oligomerize unreacted ethylene. The second-stage oligomerization reactors may be run at a temperature from about 80° C. (176° F.) to about 180° C. (356° F.) and at a pressure from about 2.1 MPa (300 psig) to about 7.6 MPa (1100 psig).

The first-stage oligomerization catalyst may include a zeolitic catalyst. The zeolite may comprise between about 5 and about 95 wt % of the catalyst, for example between about 5 and about 85 wt %. Suitable zeolites have a framework with a ten-ring pore structure such as TON, MTT, MFI, MEL, AFO, AEL, EUO and FER. The second-stage oligomerization catalyst is preferably an amorphous silica-alumina base with a metal from either Group VIII and/or Group VIB in the periodic table. In an aspect, the catalyst has a Group VIII metal promoted with a Group VIB metal. Nickel is preferably the Group VIII metal. Typically, the silica and alumina will only be in the base, so the silica-to-alumina ratio will be the same for the catalyst as for the base. The metals can either be impregnated onto, co-mulled or ion exchanged with the silica-alumina base. Catalysts for oligomerization may have a Low Temperature Acidity Ratio of at least about 0.15, suitably of about 0.2, and preferably greater than about 0.25, as determined by Ammonia Temperature Programmed Desorption (Ammonia TPD). Additionally, a suitable catalyst will have a surface area of between about 50 and about 400 m²/g as determined by nitrogen BET. It is contemplated that the zeolitic catalyst specified for the first stage oligomerization catalyst can be used as the second stage oligomerization catalyst and the metal on amorphous silica-alumina may be used as the first stage oligomerization catalyst.

The second stage oligomerate may be fed to a depropanizer column to remove C3− hydrocarbons in the overhead from C4+ olefins in the bottoms. The depropanizer column may be operated at a bottom temperature of about 194° C. (381° F.) to about 333° C. (630° F.) and an overhead pressure of about 207 kPa (gauge) (30 psig) to about 1.14 MPa (gauge) (165 psig).

The C4+ olefinic bottom stream may be fed to an olefin splitter column to split C4-C8 olefins for recycle to the oligomerization reactors while C9+ olefins are transported to the hydrogenation section. The olefin splitter column may be operated at a bottom temperature of about 200° C. (400° F.) to about 315° C. (600° F.) and an overhead pressure of about 35 kPa (gauge) (5 psig) to about 420 kPa (gauge) (60 psig).

The olefins splitter bottom stream comprising C9+ olefins are combined with the fourth hydrogen stream in line 146 and charged to a hydrogenation reactor to saturate the olefins for usage as fuels. Charge to the hydrogenation reactor occurs at 125° C. (257° F.) to about 204° C. (400° F.) and 3.5 MPa (500 psig) to about 6.9 MPa (1000 psig). An excess of hydrogen may be employed to ensure complete saturation such as about 1.5 to about 2.5 of stochiometric hydrogen. The hydrogenation catalyst may be a noble metal on alumina comprising about 0.5 to about noble metal. The hydrogenated effluent may be separated and the liquid hydrogenated effluent may be stripped of light gases in a stripping column operated at a bottoms temperature of about 232° C. (450° F.) to about 316° C. (600° F.) and an overhead pressure of about 207 kPa (30 psig) to about 689 kPa (100 psig). A stripped bottoms stream may be sent to a product fractionation column to provide a jet fuel stream in a side line 182 and a diesel stream in a bottoms line 184. Some of the diesel stream may be recycled as diluent to the oligomerization section. The product fractionation column may be operated at a bottom temperature of about 288° C. (550° F.) to about 371° C. (700° F.) and an overhead pressure of about 35 kPa (5 psig) to about 350 kPa (50 psig).

From only renewable sources jet fuel and diesel can be produced with no net emission of carbon dioxide resulting in fuel production at low carbon intensity.

### Specific Embodiments

While the following is described in conjunction with specific embodiments, it will be understood that this description is intended to illustrate and not limit the scope of the preceding description and the appended claims.

A first embodiment of the disclosure is a process for production of a liquid fuel, comprising producing a first olefin stream in a dehydration unit producing a second olefin stream in a MTO unit; and oligomerizing the first olefin stream and the second olefin stream with an oligomerization catalyst to produce an oligomerized olefin stream comprising a fuel stream. An embodiment of the disclosure is one, any or all of prior embodiments in this paragraph up through the first embodiment in this paragraph further comprising reacting the oligomerized olefin stream with a hydrogen stream in the presence of a hydrogenation catalyst in a hydrogenation section to saturate the olefins to paraffins to produce the fuel stream. An embodiment of the disclosure is one, any or all of prior embodiments in this paragraph up through the first embodiment in this paragraph further comprising producing a C2 olefin stream in the MTO unit; combining the C2 olefin stream from the MTO unit with the first olefin stream from the dehydration unit to form a combined first olefin stream; and oligomerizing the combined first olefin stream. An embodiment of the disclosure is one, any or all of prior embodiments in this paragraph up through the first embodiment in this paragraph wherein the combined first olefin stream comprises about 5% to about 95% of the first olefin stream from the dehydration unit. An embodiment of the disclosure is one, any or all of prior embodiments in this paragraph up through the first embodiment in this paragraph further comprising reacting a carbon dioxide stream and a hydrogen stream to produce a methanol stream; contacting the methanol stream with an MTO catalyst in a MTO reactor to produce an olefin stream; and separating the olefin stream to provide the second olefin stream and the C2 olefin stream. An embodiment of the disclosure is one, any or all of prior embodiments in this paragraph up through the first embodiment in this paragraph wherein the hydrogen stream is produced from electrolysis of a water stream. An embodiment of the disclosure is one, any or all of prior embodiments in this paragraph up through the first embodiment in this paragraph wherein the water stream is produced in the dehydration unit. An embodiment of the disclosure is one, any or all of prior embodiments in this paragraph up through the first embodiment in this paragraph further comprising producing alcohol and carbon dioxide from biomass and providing the carbon dioxide stream from the production of alcohol and carbon dioxide. An embodiment of the disclosure is one, any or all of prior embodiments in this paragraph up through the first embodiment in this paragraph further comprising oligomerizing the first olefin stream and the second olefin stream over a first-stage oligomerization catalyst to provide an oligomerate stream and oligomerizing the oligomerate stream over a second-stage oligomerization catalyst to produce the oligomerized olefin stream. An embodiment of the disclosure is one, any or all of prior embodiments in this paragraph up through the first embodiment in this paragraph further comprising fermenting material made from biomass to produce the alcohol stream and the carbon dioxide stream. An embodiment of the disclosure is one, any or all of prior embodiments in this paragraph up through the first embodiment in this paragraph further comprising charging the alcohol stream to the dehydration unit to produce the first olefin stream; and removing carbon monoxide from the first olefin stream to purify the first olefin stream. An embodiment of the disclosure is one, any or all of prior embodiments in this paragraph up through the first embodiment in this paragraph wherein the MTO unit and/or the dehydration unit provide water to an electrolyzer via a water line. An embodiment of the disclosure is one, any or all of prior embodiments in this paragraph up through the first embodiment in this paragraph wherein the hydrogen stream for the hydrogenation section in the oligomerization unit is taken from the electrolyzer.

A second embodiment of the disclosure is a process for production of a liquid fuel, comprising taking a first olefin stream from a dehydration unit; taking a second olefin stream from a MTO unit; oligomerizing the first olefin stream and the second olefin stream with an oligomerization catalyst to produce an oligomerized olefin stream; and reacting the oligomerized olefin stream with a hydrogen stream in the presence of a hydrogenation catalyst to saturate the olefins to paraffins to produce a fuel stream comprising jet fuel. An embodiment of the disclosure is one, any or all of prior embodiments in this paragraph up through the second embodiment in this paragraph further comprising producing a C2 olefin stream in the MTO unit; combining the C2 olefin stream from the MTO unit with the first C2 olefin stream from the dehydration unit to form a combined first olefin stream; and oligomerizing the combined first olefin stream. An embodiment of the disclosure is one, any or all of prior embodiments in this paragraph up through the second embodiment in this paragraph wherein the combined first olefin stream comprises from about 25% to about 95% of the first olefin stream. An embodiment of the disclosure is one, any or all of prior embodiments in this paragraph up through the second embodiment in this paragraph further comprising reacting a carbon dioxide stream and a hydrogen stream to produce methanol; contacting the methanol with an MTO catalyst in a MTO reactor to produce an olefin stream; and separating the olefin stream to provide the second olefin stream and the C2 olefin stream. An embodiment of the disclosure is one, any or all of prior embodiments in this paragraph up through the second embodiment in this paragraph further comprising producing ethanol and carbon dioxide from biomass and providing the carbon dioxide stream from the production of ethanol.

A third embodiment of the disclosure is a process for production of a liquid fuel, comprising taking a first olefin stream from a dehydrated alcohol stream; taking a second olefin stream from a MTO reactor effluent stream; oligomerizing the first olefin stream and the second olefin stream with an oligomerization catalyst to produce an oligomerized olefin stream; and reacting the oligomerized olefin stream with a hydrogen stream in the presence of a hydrogenation catalyst to saturate the olefins to paraffins to produce a fuel stream. An embodiment of the disclosure is one, any or all of prior embodiments in this paragraph up through the third embodiment in this paragraph further comprising producing a C2 olefin stream in the MTO unit; combining the C2 olefin stream with the first olefin stream to form a combined C2 olefin stream; and oligomerizing the combined C2 olefin stream. Without further elaboration, it is believed that using the preceding description that one skilled in the art can utilize the present invention to its fullest extent and easily ascertain the essential characteristics of this invention, without departing from the spirit and scope thereof, to make various changes and modifications of the invention and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

1. A process for production of a liquid fuel, comprising:
producing a first olefin stream in a dehydration unit
producing a second olefin stream in a MTO unit; and
oligomerizing said first olefin stream and said second olefin stream with an oligomerization catalyst to produce an oligomerized olefin stream comprising a fuel stream.

2. The process of claim 1 further comprising reacting said oligomerized olefin stream with a hydrogen stream in the presence of a hydrogenation catalyst in a hydrogenation section to saturate the olefins to paraffins to produce said fuel stream.

3. The process of claim 1 further comprising:
producing a C2 olefin stream in the MTO unit;
combining said C2 olefin stream from the MTO unit with said first olefin stream from the dehydration unit to form a combined first olefin stream; and
oligomerizing said combined first olefin stream.

4. The process of claim 3 wherein said combined first olefin stream comprises about 5% to about 95% of said first olefin stream from the dehydration unit.

5. The process of claim 3 further comprising:
reacting a carbon dioxide stream and a hydrogen stream to produce a methanol stream;
contacting said methanol stream with an MTO catalyst in a MTO reactor to produce an olefin stream; and
separating said olefin stream to provide said second olefin stream and said C2 olefin stream.

6. The process of claim 5 wherein the hydrogen stream is produced from electrolysis of a water stream.

7. The process of claim 6 wherein the water stream is produced in the dehydration unit.

8. The process of claim 5 further comprising producing alcohol and carbon dioxide from biomass and providing said carbon dioxide stream from the production of alcohol and carbon dioxide.

9. The process of claim 1 further comprising oligomerizing said first olefin stream and said second olefin stream over a first-stage oligomerization catalyst to provide an oligomerate stream and oligomerizing said oligomerate stream over a second-stage oligomerization catalyst to produce said oligomerized olefin stream.

10. The process of claim 8 further comprising fermenting material made from biomass to produce said alcohol stream and said carbon dioxide stream.

11. The process of claim 10 further comprising:
charging said alcohol stream to said dehydration unit to produce said first olefin stream; and
removing carbon monoxide from said first olefin stream to purify said first olefin stream.

12. The process of claim 3 wherein the MTO unit and/or the dehydration unit provide water to an electrolyzer via a water line.

13. The process of claim 12 wherein said hydrogen stream for the hydrogenation section in the oligomerization unit is taken from the electrolyzer.

14. A process for production of a liquid fuel, comprising:
taking a first olefin stream from a dehydration unit;
taking a second olefin stream from a MTO unit;
oligomerizing said first olefin stream and said second olefin stream with an oligomerization catalyst to produce an oligomerized olefin stream; and
reacting said oligomerized olefin stream with a hydrogen stream in the presence of a hydrogenation catalyst to saturate the olefins to paraffins to produce a fuel stream comprising jet fuel.

15. The process of claim 14 further comprising:
producing a C2 olefin stream in the MTO unit;
combining said C2 olefin stream from the MTO unit with said first C2 olefin stream from the dehydration unit to form a combined first olefin stream; and
oligomerizing said combined first olefin stream.

16. The process of claim 15 wherein said combined first olefin stream comprises from about 25% to about 95% of said first olefin stream.

17. The process of claim 15 further comprising:
reacting a carbon dioxide stream and a hydrogen stream to produce methanol;
contacting said methanol with an MTO catalyst in a MTO reactor to produce an olefin stream; and
separating said olefin stream to provide said second olefin stream and said C2 olefin stream.

18. The process of claim 17 further comprising producing ethanol and carbon dioxide from biomass and providing said carbon dioxide stream from the production of ethanol.

19. A process for production of a liquid fuel, comprising:
taking a first olefin stream from a dehydrated alcohol stream;
taking a second olefin stream from a MTO reactor effluent stream;
oligomerizing said first olefin stream and said second olefin stream with an oligomerization catalyst to produce an oligomerized olefin stream; and
reacting said oligomerized olefin stream with a hydrogen stream in the presence of a hydrogenation catalyst to saturate the olefins to paraffins to produce a fuel stream.

20. The process of claim 19 further comprising:
producing a C2 olefin stream in the MTO unit;
combining said C2 olefin stream with said first olefin stream to form a combined C2 olefin stream; and
oligomerizing said combined C2 olefin stream.
